(19) United States
(12) Patent Application Publication
(10) Pub. No.: US 2013/0310730 A1
(43) Pub. Date: Nov. 21, 2013

(54) Methods And Compositions For Administering A Specific Wavelength Phototherapy

(71) Applicant: Applied Biology, Inc., Irvine, CA (US)

(72) Inventors: Andy Ofer Goren, Newport Beach, CA (US); John McCoy, Downey, CA (US)

(73) Assignee: Applied Biology, Inc., Irvine, CA (US)

(21) Appl. No.: 13/948,090

(22) Filed: Jul. 22, 2013

Related U.S. Application Data
(63) Continuation-in-part of application No. 13/669,435, filed on Nov. 5, 2012.
(60) Provisional application No. 61/555,130, filed on Nov. 3, 2011.

(51) Int. Cl. A61N 5/06; A61K 8/44; A61K 8/49

(52) U.S. Cl. CPC A61N 5/06; A61K 8/498; A61K 8/445
USPC 604/20; 424/60

(57) ABSTRACT

Methods are disclosed for administering electromagnetic radiation (EMR), which may include filtering EMR from part of the EMR spectrum while allowing passage of EMR at a desired wavelength. A fluorescent component may be included, which absorbs EMR at one wavelength and emits EMR at the desired wavelength. Uses may include the treatment of acne. Pruritus may also be treated by allowing the passage to the skin of a particular UV wavelength from sunlight, which has an immunosuppressive effect, while protecting the skin from other harmful UV radiation.

### CROSS-REFERENCE TO RELATED APPLICATIONS

This application is a continuation-in-part of U.S. application Ser. No. 13/669,435, filed Nov. 5, 2012, which claims priority to U.S. Provisional Application Ser. No. 61/555,130, filed Nov. 3, 2011, entitled “System and Method for Administering a Specific Wavelength Phototherapy,” both of which are incorporated herein in their entirety.

### BACKGROUND

1. Technical Field

The inventions described herein relate to methods and compositions for administering electromagnetic radiation (EMR), for therapeutic or cosmetic purposes, or for purposes of curing a polymeric material.

2. Description of the Related Art

Many dermatological conditions, such as vitiligo, psoriasis, atopic dermatitis, acne and pruritis show a strong response to phototherapy. Recently, narrowband-UVB (NB-UVB), a UV phototherapy that utilizes a 311 nm wavelength, or thereabouts, has been shown to be a safe and effective modality for UV phototherapy. Similarly, blue light therapy in the visible range has been shown to be clinically effective for the treatment of acne. Currently, most effective phototherapy is administered in medical offices.

While effective, compliance with NB-UVB phototherapy can be a difficult due to the time commitment required for the treatment. For example, vitiligo patients undergoing NB-UVB phototherapy may need to visit the medical office two to three times a week for at least two to three months. This significant time commitment is the main drawback to phototherapy and can affect a patient's treatment compliance. Therefore, a phototherapy alternative that patients can safely use at home would be beneficial.

Portable phototherapy lamps are available for in home use; however, applying the proper and effective dosage may be difficult and unsafe for patients. In addition, when phototherapy is administered at medical offices, an artificial light source (NB-UVB) is used. The light source emits NB-UVB at a specific therapeutic range as well as a significant amount of non-therapeutic harmful UVB. A topical agent that can reduce harmful radiation exposure at the clinic will be highly valuable for patient safety.

Vitamin D is an essential nutrient for human health that promotes the growth of bone. Vitamin D is acquired by humans in diet or endogenously synthesized with adequate sun exposure. Not all wavelengths of light promote the synthesis of vitamin D equally. Similarly, the erythema (sunburn) reaction of skin is also wavelength dependent.

Research has indicated that UVB light in the range 306-310 nm has the greatest offset of benefit for the production of vitamin D versus the negative effects of erythema. As such, a band-pass therapeutic cream that selectively passes radiation in this region would be an improvement to currently available sunscreens, which completely inhibit the endogenous synthesis of vitamin D from sun exposure.

Additionally, UV light sources are commonly used in the manufacturing industry for drying inks, coatings, adhesives and other UV sensitive materials through polymerization (curing). Selecting the right spectral output is vital for UV-curing performance. Unfortunately, UV-curing radiation sources often emit a broad spectrum of UV radiation that may contain wavelengths of light that are not beneficial to the curing process but may produce negative effects in the manufactured product (e.g. heating). As such, a UV radiation band-pass filter that could selectively pass desirable wavelengths of light would be beneficial to the use of curing in manufacturing processes.

### BRIEF SUMMARY

Described herein are methods for administering specific wavelengths of electromagnetic radiation while excluding electromagnetic radiation of other frequencies. Such methods may be used for treatment of acne, pruritus, and other therapeutic purposes, or other non-therapeutic purposes.

One embodiment described herein is a method of delivering, from a light source that emits a broad spectrum of electromagnetic radiation (EMR) including a predetermined wavelength band, a dose of EMR within the predetermined wavelength band to an object, wherein the object is covered with a covering composition that selectively allows passage of EMR within the predetermined wavelength band, comprising the steps of: providing between the light source and the object a fluorescent component that emits EMR within the predetermined wavelength band after absorbing at a shorter wavelength within the broad spectrum emitting from the light source; and exposing the fluorescent component and the object to the light source, whereby the object is exposed to EMR from the light source within the predetermined wavelength band, and EMR emitted by the fluorescent component.

Another embodiment described herein is a kit comprising: a broad-spectrum sunscreen with a sun protection factor (SPF) of at least 15; a topical cream or spray, suitable for application to human skin, comprising a fluorescent component that emits EMR within a predetermined wavelength band after absorbing at a shorter wavelength within the broad spectrum of sunshine, wherein the sunscreen allows passage of EMR at the predetermined wavelength.

Another embodiment described herein is a method for treating a human subject with chronic or acute pruritus by delivering a dose of ultraviolet radiation to affected skin of the subject, comprising the steps of: covering the affected skin with a topical composition which selectively allows passage of one predetermined band of EMR within the UVA or UVB range (about 290 nm to about 400 nm), while excluding EMR of wavelengths shorter than said band; and exposing the affected skin to solar radiation until the affected skin has received a dose of EMR within the predetermined band effective to induce immunosuppression in the skin of the subject.

Other embodiments are disclosed herein.

### BRIEF DESCRIPTION OF THE DRAWINGS

The accompanying drawings, which are incorporated into this specification, illustrate one or more exemplary embodiments of the inventions disclosed herein and, together with the detailed description, serve to explain the principles and exemplary implementations of these inventions. One of skill in the art will understand that the drawings are illustrative only, and that what is depicted therein may be adapted based on the text of the specification or the common knowledge within this field.

In the drawings, where like reference numerals refer to like reference in the specification:

FIG. 1 is a flowchart showing a method of applying a photocream.

FIG. 2 shows an example of a computerized system for conducting or analyzing an assay to test DNA samples and providing a result.

FIG. 3 is an example of absorption spectra of photocream containing 0.75% (w/w) Silymarin (CAS #22888-70-6) and 1.125% (w/w) diethylamino hydroxybenzoyl hexyl benzoate (CAS #302776-68-7) applied at a thickness of 20 μm.

FIG. 4 shows a transmittance profile for a band-pass photocream.

FIG. 5 is a representation of an example of wavelength dependent erythema weighted irradiance.

FIG. 6 shows an example UV transmittance spectrum of a photocream formulated with 2% (w/w) Silymarin (CAS #22888-70-6), when applied at a thickness of 20 μm.

FIG. 7 shows an example absorption spectrum of a photocream containing 1% (w/w) Silymarin (CAS #22888-70-6) and 2.5% (w/w) diethylamino hydroxybenzoyl hexyl benzoate (CAS #302776-68-7), when applied at a thickness of 20 μm.

FIG. 8 shows a transmittance profile for a band-pass photocream determined from the UV absorption spectrum of FIG. 7.

### DETAILED DESCRIPTION

The description herein is provided in the context of a system and method for administering a phototherapy. Those of ordinary skill in the art will realize that the following detailed description is illustrative only and is not intended to be in anyway limiting. Other embodiments will readily suggest themselves to such skilled persons having the benefit of this disclosure. Reference will now be made in detail to implementations as illustrated in the accompanying drawings. The same reference indicators will be used throughout the drawings and the following detailed description to refer to the same or like parts.

In the interest of clarity, not all of the routine features of the implementations described herein are shown and described. In the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

As used herein, the term UVB refers to electromagnetic radiation in the range of about 290-320 nm, while UVA refers to radiation in the range of about 320-400 nm. The boundaries of these regions are sometimes slightly varied from these numbers in the literature.

In one embodiment disclosed herein, a band-pass photocream is used to selectively filter radiation in the UVB region of the electromagnetic spectrum. The chemical composition of the photocream may be such that it absorbs wavelengths of light that are non-beneficial to the treatment of the aforementioned skin ailments. Simultaneously, the band-pass cream may selectively pass wavelengths of radiation that are beneficial for treatment. Application of the photocream is followed by exposure to either natural (sun) or artificial light. In various alternative embodiments, the filtering mechanism can be in the form of a topical agent, a film, an article of clothing, a lens, a window glass, or other light filtration mechanism having an equivalent effect.

After application of the photo-filtration device, a person (or other biological organism) could receive a controlled dose of phototherapy throughout the day. This would greatly reduce the inconvenience of the standard method of delivering phototherapy in medical offices. Furthermore, the band-pass photocream could be formulated into different dosages depending on the required amount of phototherapy, physiology, genetics of the user or the condition being treated.

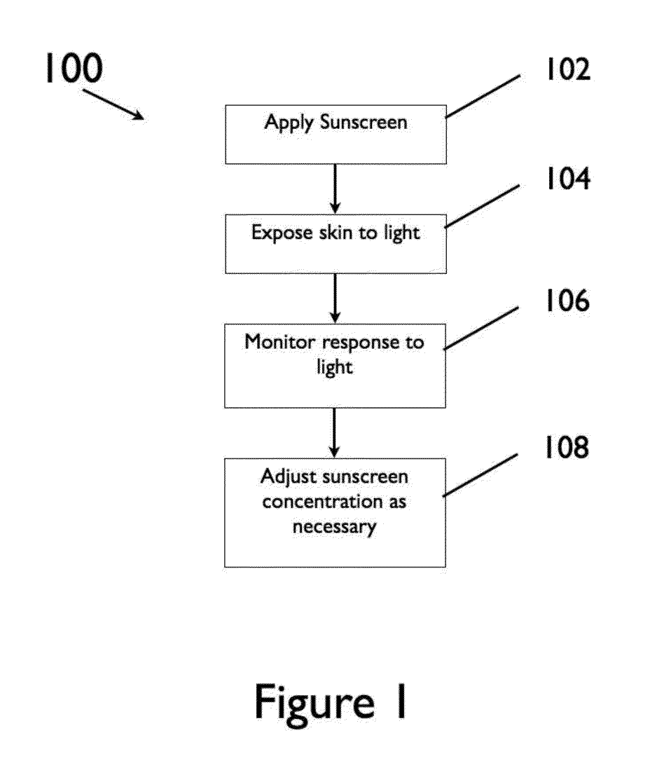

With reference to FIG. 1, a method 100 is illustrated. A band-pass photocream may be applied (102) to an exposed skin surfaces requiring phototherapy. Then, the skin surfaces may be exposed (104) to light, either as natural (sun) or artificial light. The dosage of therapeutic radiation received at the skin may be monitored (106), by the user, other personnel, or by a monitoring device such as an image-based electronic device, radiation absorption device or other method. A dosimeteter device may in one embodiment measure both therapeutic radiation and non-therapeutic radiation, or either of them separately. Furthermore, a wearable device in the form of an adhesive UV dosimeter appliqué could be used to monitor the amount of radiation exposure a person has received. The UV dosimeter appliqué could be applied to the skin prior to addition of the band-pass photocream and would itself be treated with the photocream; in another embodiment, the UV dosimeter appliqué could be treated with a polymer coating containing the same or similar (having closely related UV absorption) chemical actives as the band-pass photocream. Photocream concentration may then be adjusted (108) as necessary.

Delivery of UV light may be provided by sunlight, a UV lamp, a fluorescent tube, through amplification of available light such as through a fluorescence energy transfer reaction (FRET), or chemical, molecular, or other approaches known in the art.

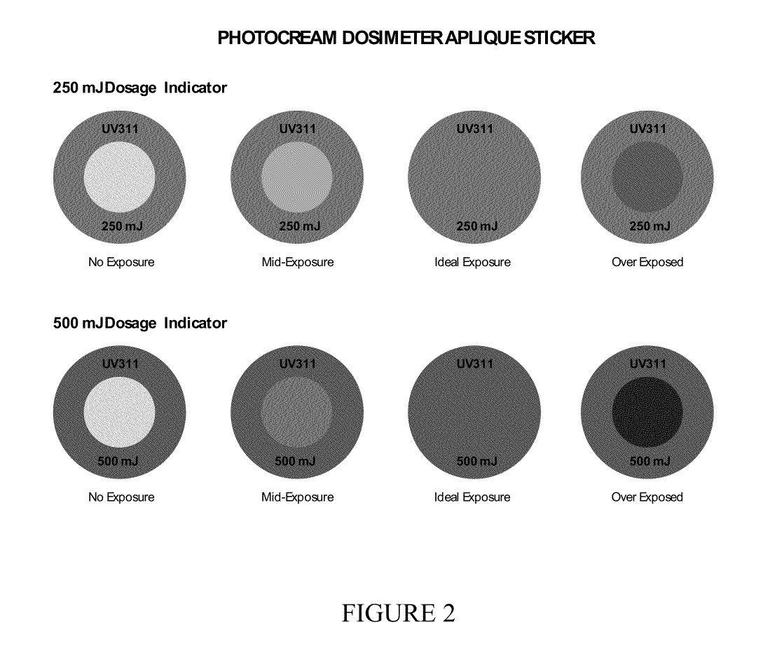

FIG. 2 illustrates an embodiment of a UV dosimeter appliqué. Two halves of a geometric shape may be used to report proper dosage of therapeutic UVB exposure. In one half of the geometric shape, a UV reactive dye may be printed. The chemistry of the dye may be such that the dye will change color in a UV dosage dependent manner. The color change of the dye may be calibrated, empirically, in a controlled laboratory environment by exposing the printed dye to a known amount of UV radiation. The empirically observed color may then be printed with standard dyes (non-UV reactive) onto the outer half of the geometric shape. This arrangement would allow for ease of use by the user in correlating color change with proper UV dosage. The UV dosimeter appliqué may be replaced with a similar device, such as a wrist band, ring or a watch.

In another embodiment of the UV dosimeter appliqué, two or more UV-reactive inks may be used to create a dosimeter that reports exposure to different bands of UV radiation. Each UV-reactive ink may have chemistry such that each ink would absorb UV radiation at separate bands (i.e. would change color based on the absorption of UV radiation at different wavelengths). As such, the system could be used to monitor exposure to UV radiation that would be considered therapeutic for a particular skin condition versus radiation that would be considered non-therapeutic. Alternatively, a therapeutic versus non-therapeutic determining dosimeter could be constructed using a broad-band UV absorbing dye that is treated with different polymer coatings containing UV absorbing actives that would filter out either therapeutic or non-therapeutic UV. The dosimeter is not limited to a chemical dosimeter, but could in one of several embodiments employ an electronic photosensor.

In yet another embodiment, a photoactive molecule may be added to the photocream; said molecule may change its chemical structure after a threshold level of UV exposure such that it would become opaque to UV radiation after receiving an appropriate dosage. As such, the added molecule would protect (block) the user from further exposure. This may be a manner in which, according to FIG. 1, the band-pass photocream concentration is adjusted (108) as required for optimum treatment benefits. The adjustments can be made based upon a database of patient conditions, treatment response, physiology, or genetics of the user and state of a device as described above in 106 or other input and/or computer analysis.

It may also be possible to use a computed analysis to select the optimum band-pass photocream concentration and/or light dosage based on the patient's response to a given concentration of the photocream with or without other characteristics of physiology or genetics of the user. According to such an approach, a method for predicting optimum photocream concentration may include: (a) constructing a N-layer neural network; (b) training the neural network with a data set of patients who have characteristics that relate to response to the photocream for the treatment of dermatological conditions, such as vitiligo, psoriasis, atopic dermatitis, etc.; (c) obtaining an image of skin response from the subject, including concentration of the photocream and light dosage; (d) generating a response-based profile from the sample, the profile being a function of values associated with a prescribed set of phototherapy parameters; (e) obtaining a difference vector from the profile; (f) inputting the difference vector into the neural network. The necessary patient data may be able to be collected from a personal device and automatically supply real time monitoring and adjustments.

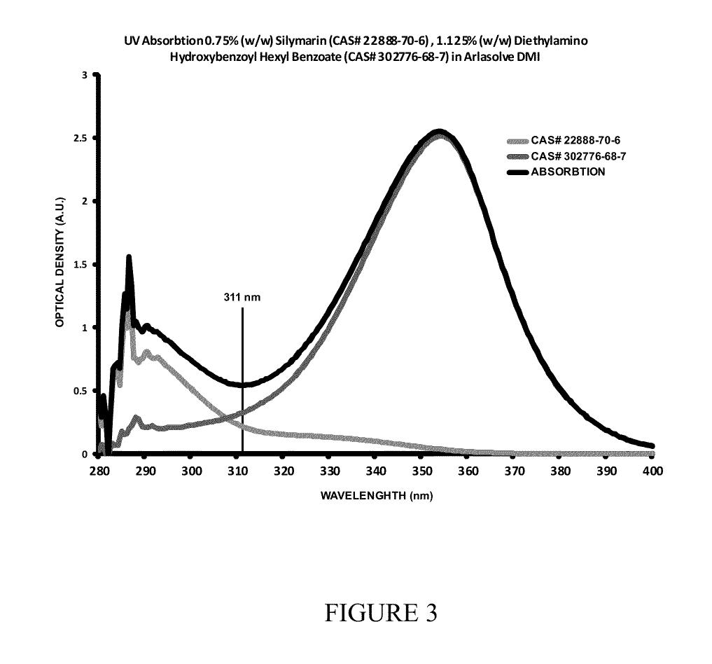

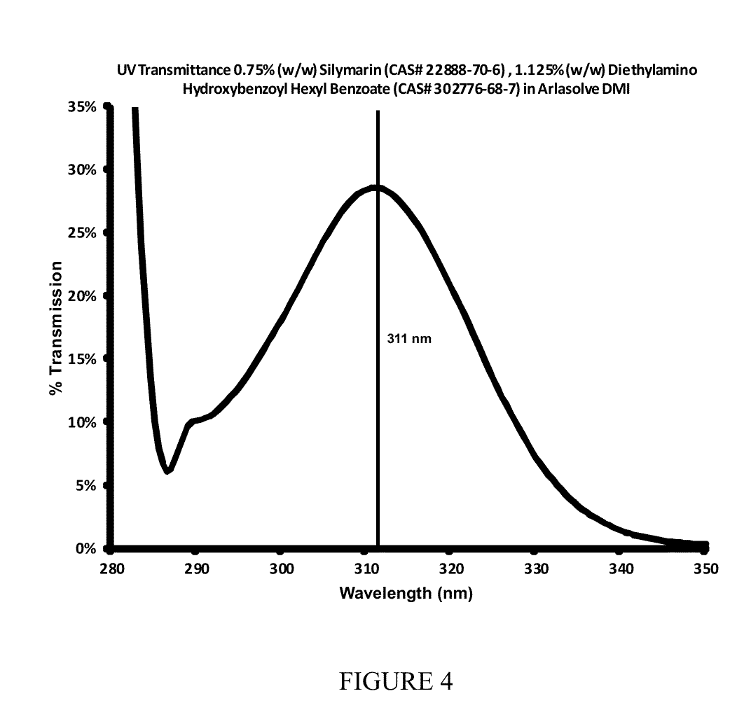

In one embodiment of the present invention, a band-pass photocream is composed such that it is optimized to have maximum transmittance at a therapeutic wavelength of 311 nm for the treatment of vitiligo, psoriasis, atopic dermatitis, and other skin conditions. Said photocream would contain two UV absorbing active ingredients having UV absorption spectra that when combined in a determined ratio would have a spectral minimum (valley) at 311 nm. For example, a band-pass photocream could be formulated with Silymarin (CAS #22888-70-6) and diethylamino hydroxybenzoyl hexyl benzoate (CAS #302776-68-7) in a weight to weight ratio of 2:3 (or less preferably within the range 1:2 to 5:6, or within the range 5:9 to 7:9) to produce an absorption spectrum with a spectral valley at 311 nm. Said photocream may contain 0.75% (w/w) Silymarin (CAS #22888-70-6) and 1.125% (w/w) diethylamino hydroxybenzoyl hexyl benzoate (CAS #302776-68-7). An illustrative absorption spectrum for such a composition is shown in FIG. 3 when applied at a thickness of 20 μm. From the UV absorption spectrum in FIG. 3, a transmittance profile for a band-pass photocream may be determined as illustrated in FIG. 4, which in this example indicates a maximum transmittance (about 29%) at 311 nm. Alternatively, a band-pass photocream could be formulated with alpha glucosyl hesperidin (CAS #161713-86-6) and diethylamino hydroxybenzoyl hexyl benzoate (CAS #302776-68-7) in the weight to weight ratio of 4:1 (or less preferably within the range 3:1 to 5:1, or within the range 7:2 to 9:2) to produce an absorption spectrum with a spectral valley at 311 nm.

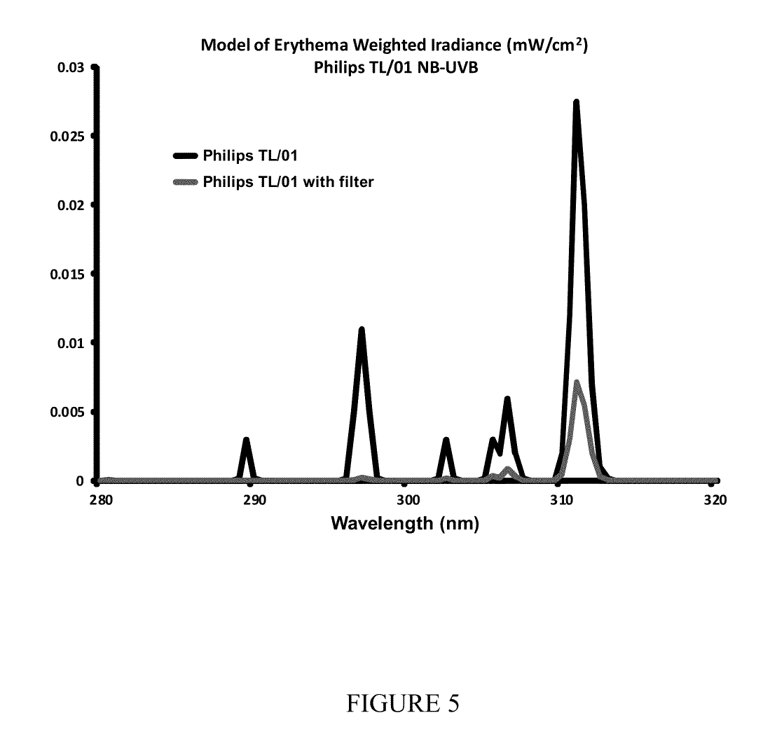

Typical light sources for the treatment of vitiligo have been reported to deliver approximately 66% of their erythema weighted irradiance in the therapeutic range 310-320 nm. The remaining erythema weighted irradiance (34%) may be delivered at wavelengths below 310 nm, which can have negative health consequence for users (e.g. erthema and cancer). An example representing the wavelength dependent erythema weighted irradiance is shown in FIG. 5.

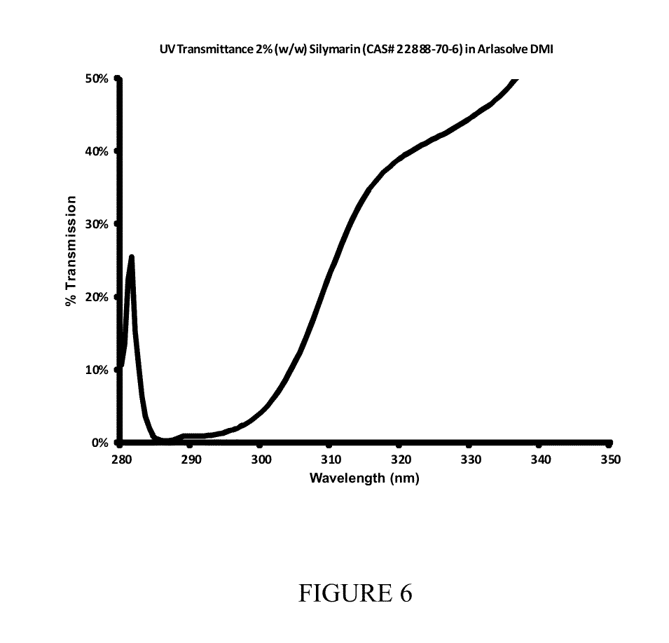

In another embodiment, a combination of UV absorbing molecules may be formulated to selectively filter non-therapeutic wavelengths of light from an artificial light source. The filtering mechanism can be in the form of a topical agent, a film, an article of clothing, a lens, or other light filtration mechanism having an equivalent effect. For example, a photocream may be formulated with 2% (w/w) Silymarin (CAS #22888-70-6) and might produce the UV transmittance spectrum in FIG. 6 when applied at a thickness of 20 μm. From the UV transmittance spectrum in FIG. 6, an adjusted erythema weighted irradiance of the Phillips TL01 (FIG. 5) may be calculated, and in this example predicts delivery of 87% of the erythema weighted irradiance in the therapeutic range 310-320 nm.

The above exemplary mode of carrying out the invention is not intended to be limiting as other methods of initiating a filter between the radiation source and radiation destination are possible. For example, a similar chemistry to the photocream described above can be incorporated into a polymer coating and applied directly to a fluorescent tube or embedded in a screen placed between the radiation source and the intended radiation destination.

In one embodiment, a band-pass therapeutic cream that selectively passes radiation in the region of UVB light in the range 306-310 nm. This region has the greatest offset of benefit for the production of vitamin D versus the negative effects of erythema. Therefore, this embodiment would provide limited protection from the deleterious effects of sun exposure (erthema) while still allowing natural synthesis of vitamin D in skin.

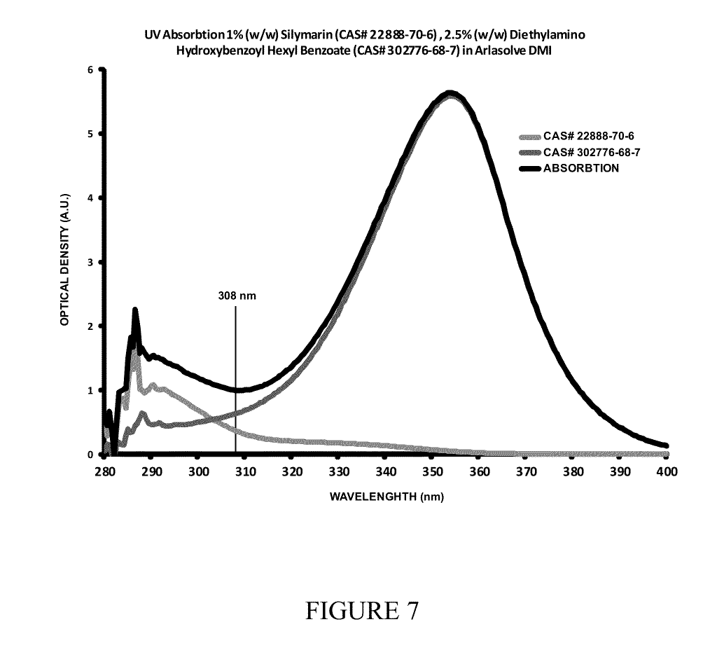

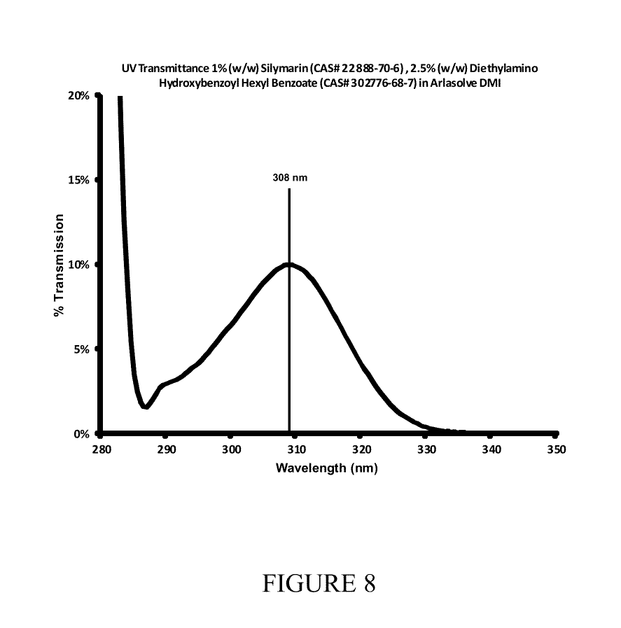

In yet another embodiment, a combination of UV absorbing molecules may be formulated to selectively pass UV-B light in the range 306-310 nm for the benefit of maximum vitamin D production while still providing limited protecting from erythema. Said photocream may contain two UV absorbing active ingredients having UV absorption spectra that when combined in a determined ratio would have a spectral minimum (valley) at 308 nm. For example, a band-pass photocream could be formulated with Silymarin (CAS #22888-70-6) and diethylamino hydroxybenzoyl hexyl benzoate (CAS #302776-68-7) in a weight to weight ratio of 2:5 (or less preferably within the range 3:10 to 1:2, or within the range 1:3 to 7:15) to produce an absorption spectrum with a spectral valley at 308 nm. Said photocream could contain 1% (w/w) Silymarin (CAS #22888-70-6) and 2.5% (w/w) diethylamino hydroxybenzoyl hexyl benzoate (CAS #302776-68-7) and might produce the absorption spectra such as that shown in FIG. 7 when applied at a thickness of 20 μm. From the UV absorption spectrum in FIG. 7, a transmittance profile for a band-pass photocream can be determined as exemplified in FIG. 8, which in this example indicates a maximum transmittance (about 10%) at 308 nm. Alternatively, a band-pass photocream could be formulated with alpha glucosyl hesperidin (CAS #161713-86-6) and diethylamino hydroxybenzoyl hexyl benzoate (CAS #302776-68-7) in a weight to weight ratio of 3:2 (less preferably a range of 5:4 to 7:4, or a range of 4:3 to 5:3) to produce an absorption spectrum with a spectral valley at 308 nm.

UV light sources are commonly used in the manufacturing industry for drying inks, coatings, adhesives and other UV sensitive materials through polymerization (curing) in lieu of evaporation. Selecting the right spectral output is vital for UV-curing performance. In general, UV-cured materials do not react the same way to UV radiation, but instead have selective responses to wavelength variations. Unfortunately, UV-curing radiation sources often emit a broad spectrum of UV radiation that may contain wavelengths of light that are not beneficial to the curing process but may produce negative effects in the manufactured product (e.g. heating). As such, a UV radiation band-pass filter that could selectively pass desirable wavelengths of light would be beneficial to the use of curing in manufacturing processes.

In yet another embodiment, a UV absorbing molecule or a combination of UV absorbing molecules may be formulated to selectively pass UV light that is most beneficial to a particular curing agent (e.g. a dye). The UV absorbing or reflective molecules could be embedded or doped into a polymeric sheet or painted on a quartz pane. These sheets may constitute a selective wavelength filter and could be used alone or combined (stacked) to achieve an appropriate band-pass filter for UV radiation. The filter may then be placed between the radiation source and the intended radiation destination. The above exemplary mode of carrying out the invention is not intended to be limiting as other methods of initiating a filter between the radiation source and radiation destination are possible. For example, a similar chemistry could be incorporated into a gel and applied directly to the intended radiation destination or the chemistry could be incorporated into a transparent mold that would benefit curing of parts normally inaccessible to light (i.e. the bottom of the mold).

Other combinations of UV absorbing actives are possible to achieve similar results to those described in the above disclosures. Examples of comparable UV absorbing active include but are not limited to: hesperidin (CAS #520-26-3), vinblastine (CAS #865-21-4), acteoside (CAS #61276-17-3), acacetin 7-O-rutinoside (CAS #480-36-4), phytoene (CAS #13920-14-4), poncirin (CAS #14941-08-3), gambogic acid (CAS #2752-65-0), chaetoglobosin (CAS #50335-03-0), poliumoside (CAS #94079-81-9), sitosteroline (CAS #474-58-8), naringin (CAS #10236-47-2), pentagalloyl glucose (CAS #14937-32-7), amentoflavone (CAS #1617-53-4), tetrandrine (CAS #518-34-3), isoacteoside (CAS #61303-13-7), (−)-phaeanthine (CAS #1263-79-2), garcinol (CAS #78824-30-3), salvianolic acid B (CAS #121521-90-2), docetaxel (CAS #114977-28-5), ecdysterone (CAS #5289-74-7), glycyrrhizic acid monosodium salt (CAS #11052-19-0), kaempferol (CAS #81992-85-0), paclitaxel (CAS #33069-62-4), silymarin (CAS #22888-70-6), isoacteoside (CAS #61303-13-7), linarin (CAS #480-36-4), pectolinarin (CAS #28978-02-1), rutin (CAS #153-18-4), kaempferol-3-O-rutinoside (CAS #17650-84-9), diosmin (CAS #520-27-4), rhoifolin (CAS #17306-46-6), avobenzone (CAS #70356-09-1), alpha glucosyl hesperidin (CAS #161713-86-6), caffeine (CAS #58-08-2), mycosporine-like amino acids, rare earth metals. Variants of these components may also be used, as well as other substances known to absorb EMR, and preferably ultraviolet light.

Alternatively, a molecule may be selected such that its absorbance maximum corresponds to the wavelength of the most therapeutic value; said molecule could then be synthesized such that a conjugated bond may be added to the molecule; in addition a second molecule would be synthesized such that a conjugated bond would be subtracted from the original molecule. In each of the synthesis schemes described above the absorption maxima of the molecule would be red-shifted or blue-shifted accordingly (i.e. increased in wavelength or decreased in wavelength). As such, an equal molar combination of the molecules would produce a filter with an absorption minimum (“valley”) at the wavelength of the absorption maximum of the original molecule.

In another embodiment, a transmissive cream or sunscreen is used which blocks out all wavelengths of light except for the wavelengths of value, or alternatively has a fluorophor that fluorescently emits light, followed with exposure to either natural (sun) or artificial light. The filtering mechanism can be in the form of a topical agent, a film, an article of cloth, a lens or other light filtration mechanism having an equivalent effect. A human or other living organism could receive a controlled dose of phototherapy throughout the day, and not be limited by the inconvenience of the standard method of delivering phototherapy in medical offices.

The transmissive cream may be used according to FIG. 1, in which, for example, a transmissive sunscreen 102 at the specific wavelength may be applied as described above. A wearable device in the form of a watch or bracelet may be used to monitor the amount of light the person has received and, in one embodiment, provide further release of filtering mechanism. Adjustments 108 may be made to the sunscreen concentration as required for optimal treatment benefits, and may be made based upon a database of patient conditions, treatment response, physiology, genetics, a monitoring device, or other input and/or computer analysis.

In another embodiment, a fluorescent molecule can be used to develop radiation at a therapeutic wavelength for a phototherapy. For example, in the case of acne phototherapy, approximately blue light (450-495 nm) may be employed. In particular, light within the wavelength of about 405-470 nm is known to have an antimicrobial effect. A molecule can be selected that would absorb light from a non-therapeutic wavelengths of the solar irradiance spectra and emit light at a therapeutic wavelength. An example of one such a molecule is 7-diethylamino-4-methylcoumarin (CAS #91-44-1), a molecule that absorbs at a maximum of 375 nm and fluoresces at a maximum wavelength of 445 nm. The fluorophore can be arranged in a cream or doped into a film or filter that can be place in-between a person (or other life) and radiation emitted from the sun or artificial light source.

In one practical arrangement of the above embodiment, a cream that blocks at least the UVA and UVB radiation (290-400 nm) but transmits visible radiation (400-700 nm) can first be applied to the skin to protect the skin from UV damage. Many such creams are commercially available as broad-spectrum sunscreens, which may for example have sun protection factors (SPFs) of 15, 30, 50, or higher. A second spray or cream could then be applied as a second layer containing a fluorescent compound such as 7-diethylamino-4-methylcoumarin. This combined application would have the properties of converting harmful UV radiation into therapeutic blue light, which would pass with endogenous blue light to the skin when exposed to solar irradiance. This exemplary mode would have the additional benefit of improving the absorbance properties (absorbance maximum) of compounds in the second applied layer.

In another embodiment, selective treatment with UVB radiation may be used to treat chronic or acute pruritus. It is known that UVB is immunosuppressive, and that pruritus may be treated by immunosuppression. UVA has also been found to be immunosuppressive. See Phan, Tai A. et al. (2006), Spectral and dose dependence of ultraviolet radiation-induced immunosuppression, Frontiers in Bioscience 11, 294-411. Therefore, pruritus may be treated by the use of a topical cream that includes a band gap allowing passage of UVA and/or UVB radiation.

A person of ordinary skill in the art will be able to determine an effective dose for immunosuppression, based on the patient's skin type and the wavelength of light used. It is typically calculated as a fraction of the minimal erythemal dose (MED), which can be derived empirically for each patient. For NB-UVB light, a typical MED would be in the range of about 100-400 mJ/cm². For broadband-UVB (BB-UVB), a typical range would be about 10-30 mJ/cm².

The above are exemplary modes of carrying out the invention and are not intended to be limiting. It will be apparent to those of ordinary skill in the art that modifications thereto can be made without departure from the spirit and scope of the invention as set forth in the following claims.

What is claimed is:

1. A method of delivering, from a light source that emits a broad spectrum of electromagnetic radiation (EMR) including a predetermined wavelength band, a dose of EMR within the predetermined wavelength band to an object, wherein the object is covered with a covering composition that selectively allows passage of EMR within the predetermined wavelength band, comprising the steps of:
providing between the light source and the object a fluorescent component that emits EMR within the predetermined wavelength band after absorbing at a shorter wavelength within the broad spectrum emitting from the light source; and
exposing the fluorescent component and the object to the light source, whereby the object is exposed to EMR from the light source within the predetermined wavelength band, and EMR emitted by the fluorescent component.

2. The method of claim 1, wherein the fluorescent component is part of the covering composition.

3. The method of claim 2, wherein the covering composition selectively allows passage of EMR at the shorter wavelength in addition to the predetermined wavelength band.

4. The method of claim 1, wherein the covering composition is part of a first layer of material, and the fluorescent component is part of a second layer on top of the first layer of material.

5. The method of claim 4, wherein the first layer is a cream or powder, and the second layer is a liquid, wherein said step of providing comprises spraying the liquid on the first layer.

6. The method of claim 1, wherein the object is the skin of a human subject, and wherein the covering composition is a topical cream or powder that blocks at least a portion of the ultraviolet light spectrum.

7. The method of claim 6, wherein said portion of the ultraviolet light spectrum comprises the UVA spectrum (about 320 nm to about 400 nm).

8. The method of claim 7, wherein said portion of the ultraviolet light spectrum comprises the UVA and UVB spectra (about 290 nm to about 400 nm).

9. The method of claim 6, wherein the human subject suffers from acne, and wherein the amount of light at the predetermined wavelength band is an effective amount for treatment of the acne.

10. The method of claim 9, wherein the predetermined wavelength band includes a range of wavelengths that include wavelengths within the range of about 405 nm to about 470 nm.

11. The method of claim 1, wherein the fluorescent component is 7-diethylamino-4-methylcoumarin (CAS #91-44-1).

12. A kit comprising:
a broad-spectrum sunscreen with a sun protection factor (SPF) of at least 15;
a topical cream or spray, suitable for application to human skin, comprising a fluorescent component that emits EMR within a predetermined wavelength band after absorbing at a shorter wavelength within the broad spectrum of sunshine, wherein the sunscreen allows passage of EMR at the predetermined wavelength.

13. A method for treating a human subject with chronic or acute pruritus by delivering a dose of ultraviolet radiation to affected skin of the subject, comprising the steps of:
covering the affected skin with a topical composition which selectively allows passage of one predetermined band of EMR within the UVA or UVB range (about 290 nm to about 400 nm), while excluding EMR of wavelengths shorter than said band; and
exposing the affected skin to solar radiation until the affected skin has received a dose of EMR within the predetermined band effective to induce immunosuppression in the skin of the subject.

14. The method of claim 13, wherein the topical composition also excludes EMR at wavelengths longer than the predetermined band.

15. The method of claim 14, wherein the predetermined band is within the UVB range (about 290 nm to about 320 nm).

16. The method of claim 15, wherein the predetermined band is a narrow band at about 311 nm, and the dose of EMR is the range of about 10 to about 30 mJ/cm².

17. The method of claim 14, wherein the predetermined band is within the UVA range (about 320 nm to about 400 nm).
